(19) United States
(12) Patent Application Publication
(10) Pub. No.: US 2010/0262193 A1
(43) Pub. Date: Oct. 14, 2010

(54) Osseosynthesis Plate with Keyhole Feature

(76) Inventors: Robert Frigg, Bettlach (CH); Tom Overes, Langendorf (CH)

Correspondence Address: Fay Kaplun & Marcin, LLP, 150 Broadway, suite 702, New York, NY, 10038, US

(21) Appl. No.: 12/754,008

(22) Filed: Apr. 5, 2010

Related U.S. Application Data
(60) Provisional application No. 61/167,598, filed on Apr. 8, 2009.

(51) Int. Cl. A61B 17/88; A61B 17/80

(52) U.S. Cl. 606/281; 606/290

(57) ABSTRACT

A bone plate comprises a first plate opening extending therethrough, the first plate opening comprising a first cylindrical portion sized and shaped to receive therethrough a shaft of a first bone fixation element to be passed through the plate to fix the bone plate to a target bone to be treated in a desired position, a top surface of the first cylindrical portion comprising a recess sized and shaped to lockingly seat a head of the first bone fixation element therein and a first elongated portion open to the first cylindrical portion and a side of the bone plate, the first elongated portion being sized and shaped to permit slidable movement of the shaft of the first bone fixation element therethrough while preventing passage therethrough of the head of the first bone fixation element.

### PRIORITY

The present application claims priority to U.S. Provisional Application Ser. No. 61/167,598 entitled “Osseosynthesis Plate With Keyhole Feature” filed on Apr. 8, 2009 to Robert Frigg and Tom Overes, the contents of which are incorporated herein by reference.

### BACKGROUND INFORMATION

The repositioning and proper alignment of bone fragments is essential to performing an effective bone fracture treatment procedure and restoring proper bone length, axis and rotation. If individual fragments of the fractured bone are misaligned, even force distribution across the bone is compromised, thus increasing of the likelihood of further injury due to excessive wear of joint surfaces, etc. due to a less than optimal load distribution. Improper alignment of the bone can also have a significant impact on motion patterns of a bone joint.

### SUMMARY OF THE INVENTION

The present invention is directed to a bone plate comprising a first plate opening extending therethrough. The first plate opening comprises a first cylindrical portion sized and shaped to receive therethrough a shaft of a first bone fixation element to be passed through the plate to fix the bone plate to a target bone to be treated in a desired position. A top surface of the first cylindrical portion comprises a recess sized and shaped to lockingly seat a head of the first bone fixation element therein and a first elongated portion open to the first cylindrical portion and a side of the bone plate. The first elongated portion is sized and shaped to permit slidable movement of the shaft of the first bone fixation element therethrough while preventing passage therethrough of the head of the first bone fixation element.

### BRIEF DESCRIPTION OF THE DRAWINGS

FIG. 1 shows a first perspective view of a bone plate according to the present invention;

FIG. 2 shows a partial zoomed view of the bone plate of FIG. 1;

FIG. 3 shows a first view of the bone plate of FIG. 1 in a first operative configuration;

FIG. 4 shows a second view of the bone plate of FIG. 1 in a second operative configuration;

FIG. 5 shows a third view of the bone plate of FIG. 1 in a third operative configuration;

FIG. 6 shows a fourth view of the bone plate of FIG. 1 in a fourth operative configuration;

FIG. 7 shows a first zoomed view of the elongating instrument of FIGS. 4-6;

FIG. 8 shows a second zoomed view of the elongating instrument of FIGS. 4-6; and

FIG. 9 shows a perspective view of a bone plate according to a first alternate embodiment of the present invention.

### DETAILED DESCRIPTION

The present invention may be further understood with reference to the following description and the appended drawings, wherein like elements are referred to with the same reference numerals. The present invention relates generally to methods and devices for the alignment and stabilization of fractured bones. Specifically, the present invention relates to methods and devices for realigning fractured portions of bone and fixing a bone plate thereover to maintain the restored configuration. An exemplary method according to the present invention permits the partial insertion of bone fixation screws into the bone prior to bone alignment and attachment of a bone plate. The reversed order system and method of the present invention provides the additional advantage of facilitating a minimally invasive surgery, as will be described in greater detail hereinafter. Although embodiments of the present invention are described with respect to long bones of the body, it is respectfully submitted that the present invention may also be employed in conjunction with a bone fixation procedure for any bone in a living body such as the spine or any other bone fixation procedures that employ minimally invasive surgery techniques. Those skilled in the art will understand that, as used in this application, the term top in this application refers to a portion of the device proximate to a physician or other user thereof while bottom refers to portions of the device separated from the user—i.e., inserted into a living body on which the device is to be employed. It is further noted that directional terms assigned to components of the present invention are used to describe an exemplary procedure and are not intended to limit the invention. That is, the device may be oriented in and employed in other directions and positions if so desired, as would be understood by those skilled in the art.

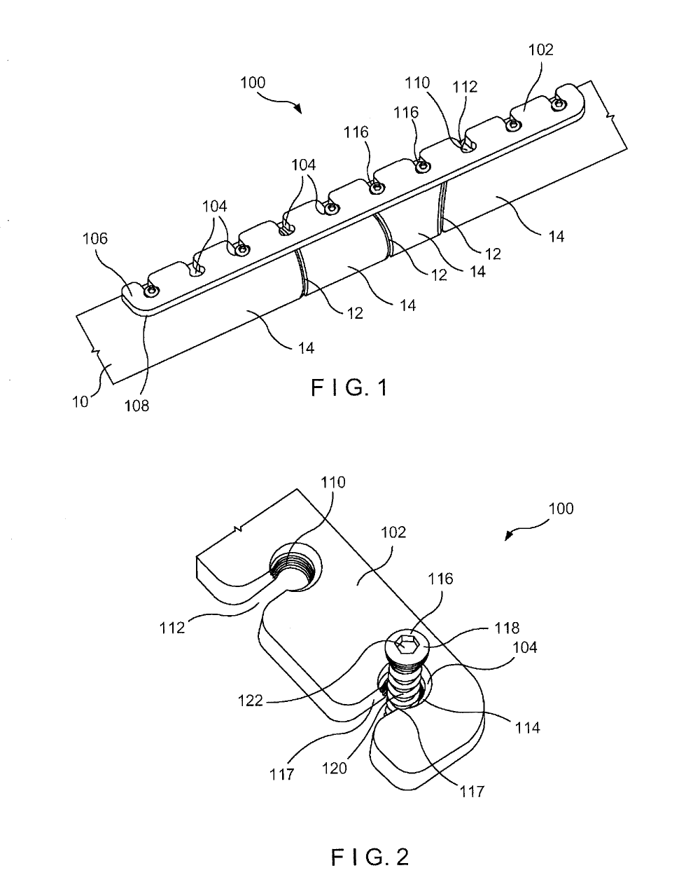

As shown in FIGS. 1-2, a device 100 according to an exemplary embodiment of the invention comprises a plate body 102 formed with a plurality of plate openings 104 extending therethrough from a top surface 106 to a bottom surface 108. Each of the plate openings 104 comprises a first portion 110 formed in a substantially cylindrical shape and a second elongated portion 112 which extends laterally therefrom through a lateral face of the plate body 102. In one embodiment, the first portion 110 may be threaded to permit engagement with a threaded shaft or head of a bone screw 116, as will be described in greater detail in an exemplary method of use of this device described in more detail below. A bevel 114 is formed adjacent a top surface of the first portion 110 to seat a head 118 of a fixation element such as a bone screw 116. The second elongate portion 112 is sized and shaped to permit only a shaft 120 of the bone screw 116 to slide therepast and prevent lateral movement of the head 118 thereinto when screwed into a locked position. Specifically, in a locked configuration, the head 118 of the bone screw 116 is seated in the bevel 114 and is prevented from moving laterally relative to the plate body 102 due to engagement with a wall 117 adjacent the bevel 114, as shown in FIG. 2. The plate body 102 may be dimensioned to conform to the anatomy and curvature of a fractured portion of bone to which it will be attached, as those skilled in the art will understand. A material of the plate body 102 can be selected from one of TAN, steel or a plastic such as polyaryletheretherketone (“PEEK”).

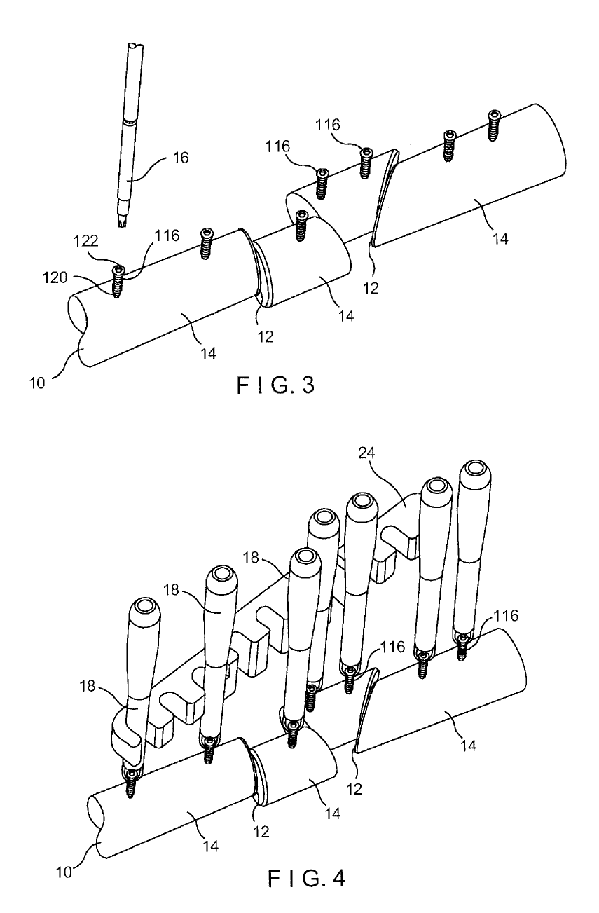

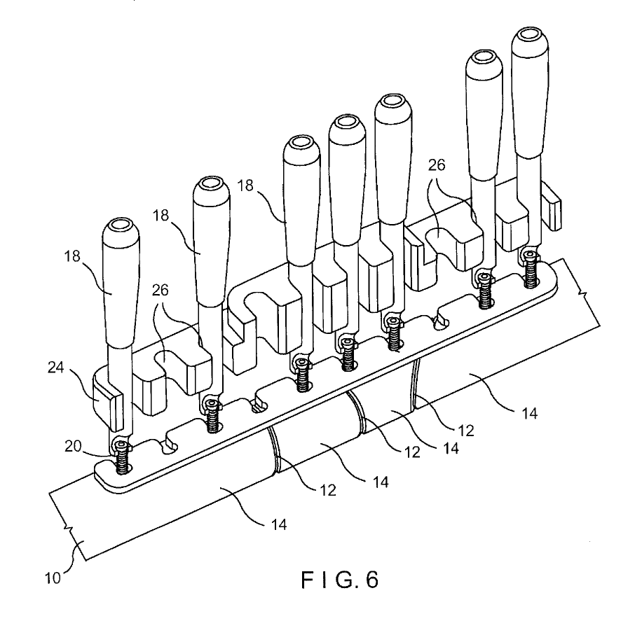

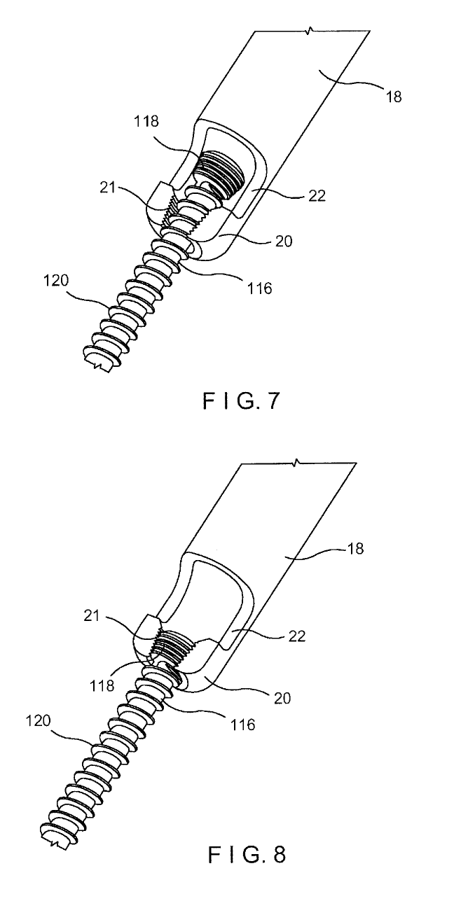

FIGS. 1 and 3-5 depict an exemplary method of use of the device 100 in treating a bone 10 with multiple fractures 12 forming separated bone fragments 14. In accordance with an exemplary minimally invasive surgery according to the present invention, a marker (not shown) that is optically or electromagnetically locatable is placed in each of the bone fragments. The markers (not shown) may be drilled into any portion of each of the bone fragments 14 except for a target bone plate 102 placement area, a depth of the drilled markers being selected to prevent movement thereof relative to the bone 10. Computed Tomography (CT) or Magnetic Resonance Imaging (MRI) is then used to generate a three-dimensional image of each of the bone fragments 14 and each of the markers (not shown). Visualization of the bone fragments 14 aids in determining an appropriate bone plate for the bone fixation procedure. It is further noted that any other visualization techniques known to those of skill in the art may be employed in preoperative planning to select an ideal plate for the procedure. Using the relationship between each marker and the corresponding bone fragment 14, a physician or other user of the device 100 screws bone a screw 116 into one or more predetermined portions of each of fragment 14, as shown in FIG. 3. Specifically, each of the bone screws 116 is positioned on the bone fragments with a longitudinal alignment of the bone screws 116 corresponding to a desired alignment in which the bone fragments 14 are to be held for healing. Each of the bone screws 116 is advanced to the bone 10 via, for example, a single minimally invasive incision formed in an portion of the skin adjacent to the bone 10. Each bone screw 116 is manipulated using a driver 16 formed with a distal tip sized and shaped to engage a recess 122 formed in the head 118 of the bone screw 116 and is screwed into the target portion of the bone 10 to a depth of approximately two-thirds of its length. Once the bone screws 116 have been screwed into each of the bone fragments 14, elongating instruments 18 are attached to each of the bone screws 116, as shown in FIG. 4. The elongating instruments 18 may be formed substantially similarly as elongation instruments used for spinal posterior fixation procedures, as those skilled in the art will understand. A bottom end 20 of each of the elongating instruments 18 is sized and shaped to engage the head 118 of a corresponding one of the bone screws 116. As shown in FIGS. 4-6, the bottom end 20 of each of the elongating instruments 18 is formed with a recess 22 sized and shaped to slidably receive the head 118 of one of the bone screws 116 therein. Once received within the recess 22, the head 118 is moved to a locked configuration in accordance with the method shown in FIGS. 7-8. Specifically, as shown in FIG. 7, the bone screw 116 is first inserted into the recess 22 so that the head 118 is located within the recess 22 and the shaft 120 is partially located within a threaded distal portion 21. A diameter of the threaded distal portion 21 is substantially the same as a diameter of the head 118. The bone screw 116 is then moved in a distal direction so that the head 118 moves into the threaded distal portion 21, threads of which are configured to engage threads formed on the head 118. The bone screw 116 is rotated in a clockwise direction to tighten the threads of the head 118 within the threaded distal portion 21, thus locking the bone screw 116 relative to the elongating instrument 18. When the elongating instrument 18 is to be disengaged from the bone screw 116, the elongating instrument is rotated in a counter-clockwise direction to unscrew the threaded distal portion 21 from the head 118.

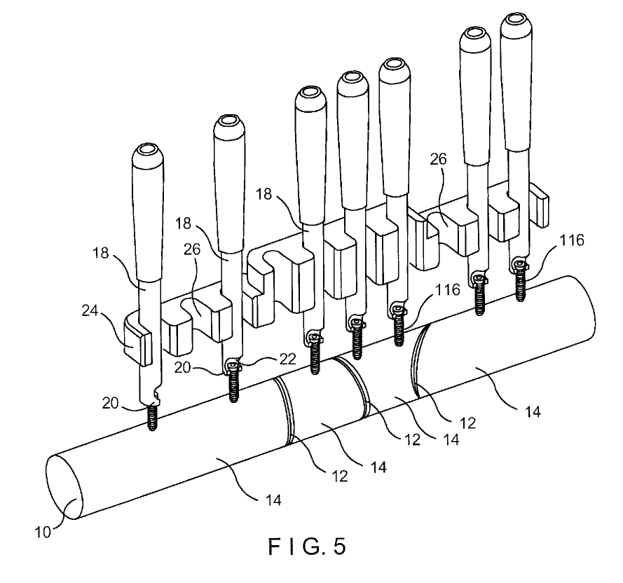

An alignment template 24 is then attached to the shafts of each of the elongating instruments 18 and the bone fragments 14 are forced into an aligned configuration, as shown in FIG. 5. The alignment template 24 remains external to the body in the operative configuration and is formed as an elongated body with a series of grooves 26 formed on a side wall thereof, the grooves 26 sized and shaped to slidably receive the shafts of the elongating instruments 18 therein and are aligned so that, when the shafts of the elongating instruments 18 are fully inserted into the grooves 26, the elongating instruments 18 and, consequently, the bone screws 116 are in a desired alignment with one another (e.g., aligned along a common axis or along a predetermined path). In this position, the bone fragments 14 are in a desired spatial relationship with one another. The elongating instruments 18 engage the grooves 26 with a substantial friction fit to prevent movement of the template 24 relative thereto when in the engaged configuration as shown in FIG. 5. Once the bone fragments 14 have been positioned in the desired spatial relationship to one another, the bone plate 102 is inserted into the body through a minimally invasive incision formed adjacent the fracture site, wherein the minimally invasive incision is only slightly larger than a width of the bone plate 102. The elongating instruments 18 are inserted through stab incisions formed adjacent individual target location thereof, as those skilled in the art will understand. The bone plate 102 is then slid over the aligned bone 10 such that each of the plate openings 104 slidably receives a corresponding one of the bone screws 116. A first one of the elongating instruments 18 is then disengaged from a first one of the bone screws 116 and the driver 16 is used to screw the first bone screw 116 completely into the bone 10 until the head 118 engages the bevel 114 of the corresponding plate hole 104. In this position, a position of the bone screw 116 is locked relative to the bone plate 102. This step is then repeated for each of the elongating instruments 18 until each of the bone screws 116 has been screwed into and locked against the plate body 102.

It is noted that although the present invention has been described with elongating instruments 18, any other known device may be used to stabilize and reposition the fractured bone without deviating from the spirit and scope of the present invention. For example, Kirschner wires can be positioned in holes drilled into predetermined portions of the bone, as described in greater detail earlier. The Kirschner wires may then be manipulated to align the bone fragments. Cannulated screws (not shown) can be guided over each of the Kirschner wires and screwed through the bone plate and into the bone.

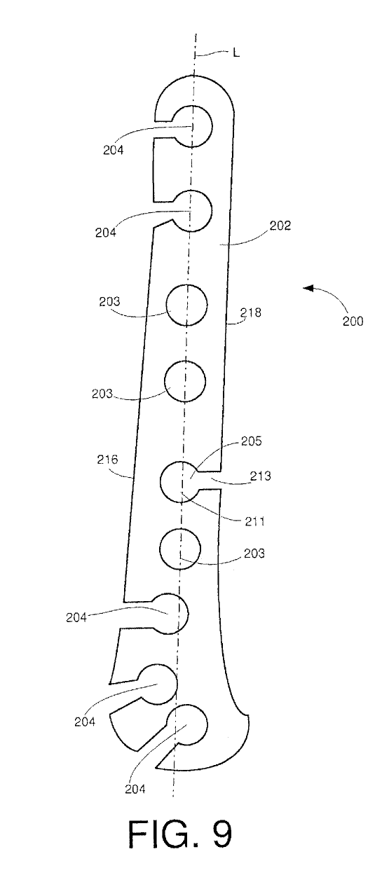

FIG. 9 shows a system 200 according to a first alternate embodiment of the present invention wherein a bone plate 202 is sized and shaped to permit fixation of a humerus (not shown). The bone plate 202 is formed with first plate openings 204 each of which comprises a first portion 210 and a second elongated portion 212 similar to the plate openings 104 of FIGS. 1-6. The bone plate 202 also comprises second plate openings 203 that are substantially cylindrical and are bound on all sides by the bone plate 202. The employment of second plate openings 203 provides an additional bone fixation means allowing a bone screw to be inserted therethrough and into the humerus in the conventional manner after the bone plate 202 has been positioned and locked down using bone screws 116 and the first plate openings 204 in the same manner described above for the bone plate 102, as those skilled in the art will understand. As seen in FIG. 9, the bone plate 202 includes, in addition to the first and second plate openings 204, 203, a third plate opening 205 which opens to a second lateral surface 216 of the plate 202 opposite the first lateral face 216 to which the first plate openings 204 open. That is, the first plate openings 204 open facing one side of a longitudinal axis L of the plate 202, while the third plate opening 205 opens toward the opposite side of the longitudinal axis L when viewed from the top surface of the bone plate 202 as shown in FIG. 9.

To apply the bone plate 202, a user applies bone screws 116 to bone fragments at locations corresponding to the first and third openings 204, 205, respectively, in the same manner described above in regard to the plate 102. The bone screws 116 may be positioned on fragments of the bone adjacent to respective first and second lateral surfaces 216, 218. The user then applies an elongating instrument 18 to each of the bone screws 116 corresponding to the first openings 204 in the same manner described above and draws these bone screws 116 into an alignment template (not shown) corresponding to the plate 202 in the same manner described above. Specifically, grooves of the alignment template (not shown) are positioned to correspond to the positions of each of the first and third openings 204, 205 in the bone plate 202 to permit the slidable insertion of the elongating instruments 18 thereinto. The user first positions the bone plate 202 over the target portion of the bone and draws the elongating instrument 18 attached to the bone screw 116 located adjacent the first lateral surface 216 into respective grooves of the alignment template (not shown). This movement draws each of the bone screws 116 into their respective first openings 204. The elongating instrument 18 attached to the bone screws 116 of adjacent the second lateral surface 218 is then drawn into a respective groove of the alignment template (not shown) to move the bone screw 116 into the third opening 205 and position all of the bone fragments into a desired spatial relationship to one another. The screws 116 are then removed from the elongating instruments 18 and tightened to lock the bone plate 202 (and the bone fragments) in the desired position in the same manner described above in regard to the plate 102. Those skilled in the art will understand that any number of second openings 205 may be included in the plate 202.

It will be apparent to those skilled in the art that various other modifications and variations may be made in the structure and the methodology of the present invention, without departing from the spirit or scope of the invention. Thus, it is intended that the present invention cover modifications and variations of the invention provided that they come within the scope of the appended claims and their equivalents.

What is claimed is:

1. A bone plate, comprising:
a first plate opening extending therethrough, the first plate opening comprising a first cylindrical portion sized and shaped to receive therethrough a shaft of a first bone fixation element to be passed through the plate to fix the bone plate to a target bone to be treated in a desired position, a top surface of the first cylindrical portion comprising a recess sized and shaped to lockingly seat a head of the first bone fixation element therein and a first elongated portion open to the first cylindrical portion and a side of the bone plate, the first elongated portion being sized and shaped to permit slidable movement of the shaft of the first bone fixation element therethrough while preventing passage therethrough of the head of the first bone fixation element.

2. The bone plate of claim 1, wherein the bone plate defines a bottom surface which, when the bone plate is placed on a target bone in a desired position, faces the bone, a top surface which, in the desired position, faces away from the bone and a first lateral surface connecting the top and bottom surfaces, the first cylindrical portion extending through the top and bottom surfaces and the first elongated portion extending through the top and bottom surfaces and being open to the first lateral surface.

3. The bone plate of claim 1, further comprising:
a second plate opening with a second cylindrical portion sized and shaped to receive therethrough a shaft of a second bone fixation element to be passed through the bone plate to fix the bone plate to a target bone to be treated in a desired position, wherein a top surface of the second cylindrical portion comprises a recess sized and shaped to lockingly seat a head of the second bone fixation element therein; and a second elongated portion sized and shaped to permit slidable movement of the shaft of the second bone fixation element therethrough while preventing passage therethrough of the head of the second bone fixation element.

4. The bone plate of claim 2, wherein the second elongated portion of the second plate opening opens in a direction facing a side of a longitudinal axis of the bone plate opposite a side of the longitudinal axis to which the first elongated portion opens through the first lateral surface.

5. The bone plate of claim 4, wherein the second elongated portion opens through a second lateral surface of the bone plate on a side of the longitudinal axis opposite that of the first lateral surface.

6. The bone plate of claim 5, wherein the first and second bone fixation elements are selected from the group consisting of a bone screw, a rod and a pin.

7. The bone plate of claim 5, wherein the first and second plate openings are separated from one another along the longitudinal axis.

8. The bone plate of claim 1, wherein the first cylindrical portion is threaded.

9. The bone plate of claim 1, wherein the first elongated portion is not threaded.

10. The bone plate of claim 5, further comprising:
a third plate opening with a third cylindrical portion sized and shaped to receive therethrough a shaft of a third bone fixation element to be passed through the plate to fix the plate to a target bone to be treated in a desired position, wherein the third plate opening is circumferentially bound by the bone plate.

11. A system for treating bone, comprising:
a bone plate including
a first plate opening extending therethrough, the first plate opening comprising a first cylindrical portion sized and shaped to receive therethrough a shaft of a first bone fixation element to be passed through the plate to fix the bone plate to a target bone to be treated in a desired position, a top surface of the first cylindrical portion comprising a recess sized and shaped to lockingly seat a head of the first bone fixation element therein and a first elongated portion open to the first cylindrical portion and a side of the bone plate, the first elongated portion being sized and shaped to permit slidable movement of the shaft of the first bone fixation element therethrough while preventing passage therethrough of the head of the first bone fixation element; and
a second plate opening with a second cylindrical portion sized and shaped to receive therethrough a shaft of a second bone fixation element to be passed through the bone plate to fix the bone plate to a target bone to be treated in a desired position, wherein a top surface of the second cylindrical portion comprises a recess sized and shaped to lockingly seat a head of the second bone fixation element therein; and a second elongated portion sized and shaped to permit slidable movement of the shaft of the second bone fixation element therethrough while preventing passage therethrough of the head of the second bone fixation element; and
an alignment template including a plurality of slots arranged so that, when coupled to the first and second bone fixation elements, moves the first and second bone fixation elements into a desired spatial relationship to one another.

12. The bone plate of claim 11, further comprising:
first and second elongating instruments configured to be connected to the first and second bone fixation elements, respectively, the first and second elongating instruments being sized and shaped so that engagement between the first and second elongating instruments and the alignment template moves the first and second bone fixation elements into the desired spatial relationship to one another.

13. A method for aligning fragments of a fractured bone, comprising:
inserting first and second bone fixation elements into first and second target portions of bone;
sliding shafts of first and second bone fixation elements into first and second plate openings, respectively, the first and second bone fixation elements being slid into first cylindrical portions of the first and second plate openings by moving shafts thereof through second elongated portions of the first and second plate openings, the second elongated portions being open to the first cylindrical portions and a side of the bone plate and being sized to prevent passage therethrough of the heads of the respective ones of the first and second bone fixation elements; and
tightening each of the first and second bone fixation elements into the bone plate to engage the first cylindrical portions of the first and second plate openings, respectively, to lock the bone plate in a desired configuration on the bone.

14. The method of claim 13, wherein each of the first and second bone fixation elements is positioned in the bone such that a longitudinal alignment of the first and second bone fixation elements in the desired spatial relationship to one another corresponds to a desired alignment of the first and second target portions of the bone.

15. The method of claim 13, further comprising:
placing markers on each of the first and second target portions of the bone to aid in the location thereof.

16. The method of claim 13, further comprising:
attaching an elongating instrument to each of the first and second bone fixation elements, the elongating instruments extending from the respective ones of the first and second bone fixation elements along longitudinal axes thereof.

17. The method of claim 16, further comprising:
engaging an aligning template with the elongating instruments to move the first and second bone fixation elements into the desired spatial relationship to one another before sliding the shafts of the first and second bone fixation elements into the first and second bone plate openings.
